(19) United States
(12) Patent Application Publication
(10) Pub. No.: US 2002/0028479 A1
(43) Pub. Date: Mar. 7, 2002

(54) Methods for the detection of fungal growth

(76) Inventors: M. Kelly Talbot, Shrewsbury, MA (US); Dawn Ecker, Worcester, MA (US)

Correspondence Address: CLARK & ELBING LLP, 176 FEDERAL STREET, BOSTON, MA, 02110-2214, US

(21) Appl. No.: 09/771,910

(22) Filed: Jan. 29, 2001

Related U.S. Application Data
(60) Provisional application No. 60/178,653, filed on Jan. 28, 2000.

(51) Int. Cl. C12Q 1/04

(52) U.S. Cl. 435/34

(57) ABSTRACT

The invention features method for quantitating the amount of living fungal cells in a culture. The method includes the steps of: a) contacting the culture with MTS and MEN so that any living fungal cells present in the culture will convert the MTS into a formazan reaction product, wherein the rate of the conversion is increased by the presence of the MEN; and b) measuring the formazan reaction product spectrophotometrically as a measure of living fungus in the culture.

### CROSS-REFERENCE TO RELATED APPLICATIONS

This application claims benefit from U.S. Provisional Application No. 60/178,653, filed Jan. 28, 2000 (now pending), which is hereby incorporated by reference.

### BACKGROUND OF THE INVENTION

The invention relates to methods of detecting fungal growth. Assays measuring cell growth have long been used as a tool to evaluate the effect of artificially altered conditions on microorganisms. These altered conditions include addition of compounds to stimulate or inhibit growth, variations of chemical and biological components of growth medium, environmental conditions such as temperature and light, and host defense cells such as human polymorphonuclear leukocytes. The simplest of these assays is visual observation of growth, with the difference between the altered and non-altered microbial cultures serving as the measure of effect.

More recently assays have been developed using an indicator which produces a measurable signal proportional to the number of living cells present in the culture. Examples of a measurable signal include the incorporation of a radioactive metabolite by the living organisms or a calorimetric signal produced by an indicator dye.

An assay with a calorimetric endpoint has the advantage of being quantitative and amenable to automation in that the assays can be read in a spectrophotometer and the data collected and calculated by a computer. This characteristic allows the assay to be performed, for example, in microtiter plates as a high throughput screen.

Levitz and Diamond (J. Infect. Dis. 152: 938-945, 1985) describe an assay in which the viability of one of several fungal organisms incubated with human white cells was measured using the tetrazolium salt MTT (3-[4,5-dimethylthiazol-2-yl]-2,5-diphenyltetrazolium) bromide.

Tellier et al. (Antimicrob. Agents Chemother. 36: 1619-1625, 1992) describe an assay using the tetrazolium salt XTT (2,3-bis [2-methoxy-4-nitro-5-sulfophenyl)-5[(phenylamino)carbonyl]-2H-tetrazolium hydroxide) as the indicator dye to measure yeast cell growth.

Meshulam et al. (J. Infect. Dis. 172: 1153-1156, 1995) used XTT as an indicator of growth of Aspergillus (A.) fumigatis.

Jahn et al. (J. Clin. Microbiol. 33: 661-667, 1995) used MTT (3-[4,5-dimethylthiazol-2-yl]-2,5-diphenyltetrazolium bromide) as an indicator of growth of Candida (C.) albicans and A. fumigatis. Freimoser et al. (Appl. Environ. Microbiol. 65: 3727-3729, 1999) also used MTT as an indicator of growth of various fungal organisms.

Assays measuring growth of filamentous fungi have been extremely difficult to perform as high throughput screens because of the growth characteristics of the organisms. These types of fungi grow very slowly and 48 hours of culture is necessary for visible evidence of growth. In addition, the growing cells often form mats of cells covering the entire surface of the culture. In these cases, a microtiter plate reader cannot be used to quantitate results because the light path of the reader is obscured by the fungal growth. In addition, dyes added to the wells often produce extremely variable results due to the lack of penetration of the dye into the cell mat.

The previously-described assays do not produce colorimetric reaction product at a rate that is rapid enough and do not provide an adequate signal-to-noise ratio for use in high throughput screens. Accordingly, there is a need for better calorimetric assays to detect fungal viability or fungal growth.

### SUMMARY OF THE INVENTION

We have discovered a method that uses a tetrazolium dye, MTS (3-[4,5-dimethylthiazol-2-yl]-5-(3-carboxymethoxyphenyl)-2-(4-sulfophenyl)-2H-tetrazolium zwitterion) and an electron coupling reagent, menadione (MEN), to detect the growth of fungi, and in particular filamentous fungi. This assay is very sensitive, and actively metabolizing cells can be reliably detected before growth is visible. The assay is more rapid than previous methods and circumvents the difficulties resulting from fungal mat formation. This assay allows the automation of antifungal assays of filamentous fungi for high throughput screening in microtiter plate formats.

Accordingly, the invention features a method for detecting or determining the amount of living fungus in a culture. The method includes the steps of: (a) contacting the culture with MTS and MEN to convert MTS into a formazan reaction product in the presence of the fungus; and (b) determining the amount of the formazan reaction product in the culture, wherein the amount of reaction product in the culture is proportional to the amount of living fungus in the culture. While the MTS is converted into the formazan reaction product in the absence of MEN, the presence of MEN increases the rate of the reaction. If the culture contains living fungus, it will cause the MTS to be converted into the formazan reaction product. If there is no live fungus, this conversion will not take place. The method is broadly applicable to a wide variety of fungi in culture, including Candida albicans, C. glabrata, C. bovinia, C. sloofii, C. parapsilosis, C. tropicalis, C. stellatoidea, C. krusei, C. pseudotropicalis, C. guilliermondi, Torulopus pintolopesii, Histoplasma capsulatum, Malassezia furfur, Sporothrix schenckii, Cryptococcus neoformans, Petriellidium boydii, Coccidioides immitis, Blastomyces dermatitidis, Paracoccidioides brasiliensis, Aspergillus flavus, A. fumigatus, A. niger, A. nidulans, A. terreus, A. clavatus, A. glaucus, Microsporum canis, M. gypseum, M. audouini, M. ferrugineum, M. fulvum, M. nanum, M. obtusa, M. vanbreuseghemii, M. persicolor, M. gallinae, M. distortum, M. cookei, Epidermophyton floccosum, Geotrichum candidum, Curvularia lunata, C. genilunata, Trichosporum beigelii, T. cutaneum, Microascus cinereus, M. manganii, M. trigonosporus, Mucor pusillus, M. circinelloides, M. ramorissimus, Rhizopus arrhizus, R. oryzae, Absidia corymbifera, A. ramosa, Basidiobolus haptosporus, Entomophthora coronata, Monosporium apiospermum, Fonsecaea pedrosoi, F. dermatitidis, F. compactum, Cladinasporium carrionii, C. bantianum, C. werneckii, Madurella mycetomi, M. grisea, Acremonium mycetoma, Leptosphaeria senegalensis, Pyenochaeta rosneroi, Phialophora jeanselmei, P. compacta, P. dermatitidis, P. gougerotii, P. parasitica, P. repens, P. richardsiae, P. spinifera, P. verrucosa, Neotestudina rosatti, Rhinosporiduim seeberi, Fusaruim solani, Trichophyton schoenleinii, T. rubrum, T. mentagrophytes, T. concentricum, T. verrucosum, T. violaceum, T. tonsurans, T. equinum, T. simii, T ajelloi, T. megninii, Phytophthora infestans, P. cinnamomi, Fusarium oxysporum, Rhizoctonia solani, Magnaporthe grisea, Penicillium digitatum, P. chrysogenum, P. citrinum, P. crustaceum, P. glaucum, P. notatum, P. patulum, P. utacale, P. roquefortii, Botrytis cinerea, Mycosphaerella graminicola, Chochliocolus carbonum, and Cercospora kikuchii.

The system of the invention can be used in a screening method for determining whether a test substance modulates (i.e., promotes or inhibits) growth of a fungus. This method includes the steps of: (a) providing a first culture and a second culture, wherein the first culture includes a fungus, culture medium sufficient for growth of the fungus, and the test substance; and the second culture includes the fungus and the culture medium but does not include the test substance; (b) culturing the first and second cultures; (c) contacting the first and second cultures with MTS and MEN to convert MTS into a formazan reaction product in the presence of any living fungus present; and (d) determining the amount of reaction product in the first and second cultures, wherein a decrease in the amount of formazan reaction product in the first culture, relative to the amount of reaction product in the second culture, identifies the test substance as a substance that inhibits growth of a fungus, and an increase in the amount of formazan reaction product in the first culture, relative to the amount of reaction product in the second culture, identifies the test substance as a substance that promotes growth of a fungus. Preferred fungi include those listed above.

In the presence of living fungus, MTS is converted into a formazan reaction product that is water soluble, allowing the present methods to be used in high throughput screens of fungal growth using, for example, microtiter plates.

Any test substance can be used in the present screening method, including naturally occurring substances and non-naturally occurring substances. Suitable test substances are small organic compounds as well as high molecular weight (150-750 daltons) substances produced by living organisms. Test substances preferably are soluble in the growth medium. Test substances can be individual compounds or libraries of compounds. One in the art will recognize that there are numerous sources for the test substance, and any of these is suitable for use in the present screening method.

The screening method of the invention can be conducted in high throughput mode. This can be achieved, for example, by culturing the fungus in multi-well or microtiter plates, or by determining the amount of reaction product spectrophotometrically.

In the methods of the invention, MTS and MEN are each added to a final concentration so that the formazan reaction product is produced to a level that is sufficient for detection by a spectrophotometer and before the fungus has grown to the extent that light cannot penetrate the culture sufficiently to be detected by a spectrophotometer. Preferably, MTS is added at a final concentration of 0.04 to 0.4 mg/ml, and MEN is added at a final concentration of 0.7 to 7 μM. At these concentrations, the reaction product can be detected in as little as one hour, although the reaction is preferably allowed to proceed for two to twenty-four hours. The culturing step that precedes the reaction step can also vary in duration. For high throughput screening, the culture step prior to the addition of the indicator is preferably one to twenty-four hours in duration.

Other features and advantages of the invention will be apparent from the following detailed description thereof, and from the claims.

### DETAILED DESCRIPTION

We have discovered a new method of detecting and quantitating viable fungal cells by measuring the transformation of MTS in the presence of MEN.

MTS is reduced by dehydrogenase enzymes found in metabolically active cells into a formazan reaction product that is water soluble. The formazan absorbs light at 450-550 nm and the amount of absorbance is directly proportional to the number of living cells in the culture. The presence of MEN in the assay enhances the signal produced, increasing the sensitivity of the assay. Thus, metabolically active cells can be detected before visible growth appears.

In general, spores or conidia of filamentous fungi are suspended in growth medium and distributed into wells of a plate (e.g., a 96-well plate). In some cases, test substances (naturally-occurring or non-naturally occurring reagents which may have stimulatory or inhibitory effects on fungal growth) are added to the wells. The plates are covered and placed in an incubator at the appropriate growth temperatures for the appropriate length of time, and then removed from the incubator for the addition of MTS and MEM. The plates are then incubated for an additional period of time (2 to 24 hours). Plates are then read at a wavelength between 450-550 nm, preferentially of 490 nm, and the data collected electronically. The assay is readily modified for the determination of growth of any filamentous fungus, including, for example, those listed above.

### EXAMPLE 1

Evaluation of Combinations of Tetrazolium Salts and Electron Doners as Indicators of growth of A. nidulans

Methods

Spores from a frozen stock of A. nidulans 200092 at 1.63×10⁷/ml) were diluted to 5×10³/ml to yield 500 spores per well in yeast extract with glucose broth supplemented with lysine and uridine.

Tetrazolium salts (listed in Table 1) were each dissolved to 1 mg/ml in phosphate-buffered saline (PBS) or water,, aliquotted, and frozen. Electron coupling agents phenazine methosulfate (PMS; lot 194-03) and menadione (MEN; lot 170-95) were prepared at 5 mM in PBS and 10 mM in acetone, respectively. Cultures had no electron coupling reagent added, PMS (120 μl of PMS per 12 ml of tetrazolium salt) or MEN (42 μl per 12 ml of tetrazolium salt). Each well received 25 μl of indicator.

Indicator (with or without electron coupling reagent) was added at the same time as spores (T0), following ˜24 hours of incubation (T24), or following ˜48 hours of incubation (T48). Wells were viewed ˜2 hours post addition. Results for T0 and T24 are provided in Table 2. NC denotes no color seen. NG denotes that when wells were viewed, spores had not germinated, unlike other wells which did not have indicator added.

TABLE 1
Tetrazolium Salts

| | | | Product | | Formazan |
|---|---|---|---|---|---|
| Compound | Abbrev. | Vendor | No. | Lot No. | Color |
| MTS Reagent | MTS | Promega | G111A | 8259201 | Brown |
| Power | | | | | |
| p-Iodonitro- | INT | Sigma | 1-8377 | 126H5042 | Orange/ |
| tetrazolium | | | | | Brown |
| Violet | | | | | |
| Tetranitro Blue | TNTB | Sigma | T-4000 | 39H50080 | Deep Blue |
| Tetrazolium | | | | | |
| Tetrazolium | BT | Sigma | T-4375 | 68H0703 | Blue |
| Blue Chloride | | | | | |
| Tetrazolium | TTC | Sigma | T-8877 | 38H1140 | Red/Purple |
| Red | | | | | |
| Tetrazolium | TV | Sigma | T-0138 | 107H1062 | Purple |
| Violet | | | | | |
| XTT | XTT | Sigma | X-4251 | 68H0694 | Orange |

TABLE 2
Results

| | | T0 24 | ˜T24 | ˜T24 |
|---|---|---|---|---|
| | | Hr Ex- | 2 Hr | 24 Hr |
| Indicator | Donor | posure | Exposure | Exposure |
| MTS Reagent Powder | None | NC | NC | NC |
| | PMS | NC | NC | Very Faint |
| | | | | Color |
| | MEN | NC/ | Dark Brown | Excellent |
| | | NG* | Color | Brown Color |
| p-Iodonitrotetrazolium | None | NC/NG | NC | Slight Pink |
| Violet | | | | Color |
| (INT) | PMS | NC/NG | Slight Pink | Slight Pink |
| | | | Color | Color |
| | MEN | NC/NG | Slight Pink | Slight Pink |
| | | | Color | Color |
| Tetranitro Blue | None | NC/NG | NC | Uneven Dark |
| Tetrazolium | | | | Grey Color |
| | PMS | NC/NG | NC | Uneven Dark |
| (TNTB) | | | | Grey Color |
| | MEN | NC/NG | Light Brown | Uneven Dark |
| | | | Color | Grey Color |
| Tetrazolium Blue | None | NC/NG | NC | Very Faint |
| Chloride | | | | Color |
| | PMS | NC/NG | NC | Very Faint |
| (BT) | | | | Color |
| | MEN | NC/NG | NC | NC |
| Tetrazolium Red | None | NC | NC | NC |
| (TTC) | PMS | NC | NC | NC |
| | MEN | NC/NG | NC | NC |
| Tetrazolium Violet | None | NC/NG | NC | Very Faint |
| (TV) | | | | Color |
| | PMS | NC/NG | NC | Very Faint |
| | | | | Color |
| | MEN | NC/NG | NC | Very Faint |
| | | | | Color |
| XTT | None | NC | NC | NC |
| | PMS | NC | NC | NC |
| | MEN | NC/NG | Light Orange | Spotty Light |
| | | | Color | Color |

Results for the T48 time-point were all unreadable because of fungal mats in the wells. When the indicators were added to the T48 wells, the solution was unable to penetrate the mat and remained as a droplet atop the mat.

The two best combinations of tetrazolium salt and electron donor were MTS with MEN and XTT with MEN. In order to validate our subjective scoring of the reagents, and to determine the extent of background associated with the assays, we compared blank samples with samples containing A. nidulans spores grown for 24 hours. The tetrazolium salts were MTS and XTT, to which were added MEN, PMS, or no electron donor. The optical was determined using a microplate spectrophotometer after 24 hours. The results are shown in Table 3.

TABLE 3
Comparison of MTS and XTT

| | T24 (MTS) | | | T24 (XTT) | | |
|---|---|---|---|---|---|---|
| | 24 Hr of Exposure | | | 24 Hr of Exposure | | |
| | No e | | | No e | | |
| | Donor | PMS | MEN | Donor | PMS | MEN |
| Blank | 0.4 | 0.485 | 0.334 | 0.056 | 0.098 | 0.055 |
| | 0.382 | 0.466 | 0.388 | 0.051 | 0.091 | 0.052 |
| | 0.327 | 0.471 | 0.393 | 0.051 | 0.089 | 0.052 |
| Avg. OD₄₅₀₋₆₅₀ | 0.370 | 0.474 | 0.372 | 0.053 | 0.093 | 0.053 |
| Spores | 0.409 | 0.478 | 1.097 | 0.079 | 0.13 | 0.469 |
| | 0.412 | 0.477 | 1.126 | 0.072 | 0.114 | 0.452 |
| | 0.371 | 0.468 | 1.115 | 0.069 | 0.12 | 0.419 |
| | 0.385 | 0.43 | 1.071 | 0.085 | 0.125 | 0.429 |
| | 0.297 | 0.52 | 1.157 | 0.08 | 0.136 | 0.448 |
| Avg. OD₄₅₀₋₆₅₀ | 0.375 | 0.475 | 1.113 | 0.077 | 0.125 | 0.443 |

Conclusions

MEN, INT, TNTB, BT, and TV each inhibit germination or growth of the spores of A. nidulans and thus should not be added at T0. Microscopically, when compared to wells that had no indicator added or had PMS added, it was evident that the spores were present but had not germinated. At 48 hours, these same wells still had not shown any signs of viability.

Following a 24 hour incubation and a two hour exposure to the indicator, several compounds (with electron donors) were able to produce formazan: MTS with MEN, INT with either PMS or MEN, TNTB with MEN, and XTT with MEN.

The wells that received indicator at 24 hours were viewed following 24 hours of exposure to indicator (Table 2). Most of the compounds were able to produce formazan as indicated by the presence of color. Several, however, exhibited no visual change and are deemed unacceptable for use in fungal growth assays: MTS without electron donor, BT with MEN, TTC with or without electron donor, and XTT without electron donor or with PMS. Several of the remaining lanes were considered light or spotty. The combination exhibiting the best results was MTS with MEN. This combination, when added to 24-hour-old cultures and allowed to incubate with the culture for an additional 24 hours produced a very dark brown, almost black color. In Table 3, it can be seen that MTS/MEN combination provided a ˜3 fold difference between the blank wells and those wells that contained spores. In contrast, the XTT/MEN combination exhibited a ˜8 fold difference between blank and spore containing wells.

### EXAMPLE 2

Further Evaluation Of MTS And MEN as an Indicator System for A. nidulans

Methods

Spores from a frozen stock of A. nidulans 200092 (at 1.63×10⁷/ml) were diluted to 5×10³/ml to yield 500 spores per well in yeast extract with glucose broth supplemented with lysine and uridine.

MTS and MEN were prepared as is described in Example 1. MEN was added at a ratio of 42 μl MEN per 12 ml of MTS. Each well received 25 μl of indicator.

Eight wells of 96 well plate received media only, while 40 wells received 500 spores per well. Spores were allowed to grow for 24 hours at 37° C. MTS/MEN solution was added and allowed to incubate for ˜2 hours.

Results

Following indicator exposure, the plate was scanned on a SpectraMax Plush® spectrophotometer from a wavelength of 350 to 750 nm with a 10 nm interval. Based on the initial scan, a second scan was performed from 400 to 600 nm with a 5 nm interval. A wavelength of 490 nm was selected and the plate was read. The results at 490 nm are shown in Table 4.

TABLE 4

| −Spores | +Spores | | | | |
|---|---|---|---|---|---|
| OD | OD | | | | |
| 0.204 | 1.089 | 1.087 | 1.076 | 1.076 | 1.187 |
| 0.201 | 1.055 | 1.029 | 1.026 | 1.021 | 1.132 |
| 0.206 | 1.041 | 1.013 | 1.002 | 1.039 | 1.113 |
| 0.197 | 1.039 | 1.005 | 1.016 | 1.07 | 1.210 |
| 0.188 | 1.006 | 1.01 | 0.998 | 1.001 | 1.045 |
| 0.188 | 1.028 | 1.064 | 1.015 | 1.054 | 1.098 |
| 0.190 | 1.041 | 1.061 | 1.002 | 1.092 | 1.077 |
| 0.193 | 1.093 | 1.099 | 1.133 | 1.084 | 1.142 |

| | | |
|---|---|---|
| Average | 0.196 | 1.062 |
| SD | 0.007 | 0.051 |
| % CV | 3.57% | 4.80% |

In Table 4, it can be seen that an OD₄₉₀ is a suitable wavelength for detection of the reaction product. Moreover, there is ˜5.4 fold difference between blank wells and those containing spores, while the % CV for both the blank wells and those with spores is low.

### EXAMPLE 3

Detection of Inhibition of A. nidulans

Methods

Spores from a frozen stock of Aspergillus nidulans 200092 (at 1.63×10⁷/ml) were diluted to 5×10³/ml to yield 500 spores per well in yeast extract with glucose broth supplemented with lysine and uridine.

MTS and MEN were prepared as is described in Example 1. MEN was added at a ratio of 42 μl MEN per 12 ml of MTS. Each well received 25 μl indicator.

98 μl of the spore suspension was added to the wells of a 96 well plate. Control wells received media only. Inhibitors, sanguinarine and clotrimazole, were added in a volume of 2 μl. Plates were allowed to incubate for 22-24 hours Plates were visually examined for MIC determination before the addition MTS/MEN solution. Plates were then incubated for 2 hours and then read at OD₄₉₀. MTS/MEN was then allowed to develop for an additional period of time up to 24 hours and the plates was re-read. Percent inhibitions and IC₅₀ values were calculated for both time points and are listed in table 5.

TABLE 5
% Inhibition

| | | 24 Hr | | 2 Hr | 24 Hr |
|---|---|---|---|---|---|
| Clotrimazole | 2 Hr | MTS/ | Sanguinarine | MTS/ | MTS/ |
| (μg/ml) | MTS/MEN | MEN | (μg/ml) | MEN | MEN |
| 64 | 86.0 | 91.9 | 200 | 7.0 | 79.5 |
| 32 | 91.5 | 92.1 | 100 | 53.3 | 87.5 |
| 16 | 94.4 | 93.4 | 50 | 77.3 | 91.7 |
| 8 | 94.4 | 94.2 | 25 | 88.4 | 92.4 |
| 4 | 95.8 | 96.2 | 12.5 | 89.4 | 93.7 |
| 2 | 93.4 | 92.3 | 6.25 | 86.9 | 86.7 |
| 1 | 94.5 | 93.9 | 3.125 | 50.8 | 42.2 |
| 0.5 | 93.0 | 92.0 | 1.5625 | 49.6 | 39.8 |
| 0.25 | 93.0 | 91.6 | 0.7813 | 45.0 | 19.7 |
| 0.125 | 94.6 | 93.6 | 0.3906 | 33.8 | 12.6 |
| 0.0625 | 86.5 | 77.0 | 0.1953 | 10.2 | -2.4 |
| 0.0313 | 56.8 | 70.1 | 0.0977 | 42.9 | 18.4 |
| 0.0156 | 26.0 | 47.2 | 0.0488 | 15.7 | 7.9 |
| 0.0078 | 35.4 | 48.5 | 0.0244 | 15.6 | 5.1 |
| 0.0039 | 15.8 | 6.6 | 0.0122 | −3.1 | −1.7 |
| | | | 0.0061 | 16.2 | 10.9 |
| Microscopic | 0.03 | ND | | 1.5625 | ND |
| MIC | | | | | |
| IC₅₀ (μg/ml) | 0.03251 | 0.00068 | | 1.421 | 2.767 |

ND: Denotes not determined due to formazan formation. Italics denotes precipitate seen.

### EXAMPLE 4

MTS and MEN as an Indicator System for Growth of other Fungal Organisms

Methods

A. fumigatis 8001 and A. niger 97-0626 spores were isolated from a 48 to dextrose agar slant and suspended to 1×10⁴/ml concentration in RPMI-MOPS media (lot 194-93) to yield 1000 spores per well. F. oxysporum spores were isolated from one week old well sporulating potato dextrose agar plate and suspended to 2.5×10⁴/ml concentration in potato dextrose broth (lot 194-193) to yield 2,500 spores per well.

MTS and MEN were prepared as is described in Example 1. MEN was added at a ratio of 42 μl MEN per 12 ml of MTS. Each well received 25 μl of indicator.

For Aspergillus sp., plates were incubated at 37° C. for ˜24 hours at which time MTS/MEN was added. Plates were read at a wavelength of 490 nm 2 hours post MTS/MEN addition. The A. fumigatis 8001 plate was allowed to incubate an additional 15 hours and was reread. For F. oxysporum, the plate was incubated at 20° C. for ˜24 hours at which time MTS/MEN was added. This plate was read following ˜17 hours of MTS/MEN exposure.

Results

The results of the assays for each of the fungal organisms are provided below.

TABLE 6
A. niger 97-0626

| No Spores | | | 1000 Spores/Well | | | | |
|---|---|---|---|---|---|---|---|
| 0.058 | 0.059 | 0.058 | 2.099 | 2.017 | 1.903 | 1.973 | 2.067 |
| 0.059 | 0.058 | 0.059 | 2.025 | 1.995 | 2.051 | 2.051 | 1.984 |
| 0.059 | 0.058 | 0.057 | 2.049 | 1.973 | 1.834 | 2.169 | 2.108 |
| 0.057 | 0.061 | 0.058 | 2.223 | 1.904 | 1.976 | 2.109 | 1.927 |
| 0.06 | 0.071 | 0.058 | 2.064 | 2.039 | 1.89 | 1.975 | 2.017 |
| 0.058 | 0.069 | 0.059 | 2.07 | 1.973 | 1.977 | 2.009 | 2.162 |

| | | |
|---|---|---|
| Average OD₄₉₀ | 0.0598 | 2.019 |
| SD | 0.0039 | 0.0874 |
| % CV | 6.5% | 4.3% |

TABLE 7
A. fumigatis 8001 Two Hours of MTS/MEN Exposure

| No Spores | | | 1000 Spores/Well | | | | |
|---|---|---|---|---|---|---|---|
| 0.06 | 0.058 | 0.056 | 1.051 | 0.943 | 0.902 | 0.968 | 0.955 |
| 0.059 | 0.058 | 0.055 | 0.988 | 0.962 | 0.883 | 0.953 | 0.91 |
| 0.06 | 0.058 | 0.057 | 0.955 | 1.03 | 0.886 | 1.029 | 0.961 |
| 0.059 | 0.058 | 0.057 | 0.99 | 0.917 | 0.965 | 1.005 | 0.955 |
| 0.06 | 0.058 | 0.058 | 0.965 | 0.94 | 1.066 | 0.932 | 1.012 |
| 0.058 | 0.056 | 0.057 | 0.951 | 0.935 | 0.912 | 0.848 | 1.043 |

| | | |
|---|---|---|
| Average OD₄₉₀ | 0.058 | 0.960 |
| SD | 0.001 | 0.052 |
| % CV | 2.4% | 5.4% |

TABLE 8
A. fumigatis 8001 Seventeen Hours of MTS/MEN

| No Spores | | | 1000 Spores/Well | | | | |
|---|---|---|---|---|---|---|---|
| 0.123 | 0.117 | 0.109 | 2.166 | 1.997 | 2.082 | 2.065 | 2.085 |
| 0.12 | 0.119 | 0.104 | 2.114 | 2.044 | 1.989 | 2.051 | 2.024 |
| 0.122 | 0.117 | 0.114 | 2.106 | 2.165 | 2.098 | 2.141 | 2.07 |
| 0.119 | 0.113 | 0.112 | 2.141 | 1.983 | 2.086 | 2.164 | 2.124 |
| 0.119 | 0.112 | 0.113 | 2.107 | 2.052 | 2.237 | 2.186 | 2.125 |
| 0.115 | 0.11 | 0.11 | 2.086 | 2.018 | 2.058 | 1.996 | 2.249 |

| | | |
|---|---|---|
| Average OD₄₉₀ | 0.115 | 2.094 |
| SD | 0.005 | 0.069 |
| % CV | 4.4% | 3.3% |

TABLE 9
F. oxysporum Seventeen Hours of MTS/MEN

| No Spores | | | 2,500 Spores/Well | | | | |
|---|---|---|---|---|---|---|---|
| 0.054 | 0.054 | 0.978 | 0.979 | 0.996 | 0.914 | 0.942 | 1.002 |
| 0.054 | 0.054 | 1.06 | 1.07 | 1.307 | 0.832 | 0.942 | 0.98 |
| 0.054 | 0.054 | 0.913 | 0.952 | 0.966 | 0.839 | 0.86 | 0.981 |

| | | |
|---|---|---|
| Average OD₄₉₀ | 0.054 | 0.973 |
| SD | 0.001 | 0.1058 |
| % CV | 1.5% | 10.9% |

TABLE 10
Fold Difference Between Blank (No Spores) and Spore Containing
Wells

| | | | Fold |
|---|---|---|---|
| Filamentous Fungi | Blank OD₄₉₀ | Spores OD₄₉₀ | Difference |
| A. fumigatis 8001 (2 Hours) | 0.058 | 0.960 | 16.6 |
| A. fumigatis 8001 (17 Hours) | 0.115 | 2.094 | 18.2 |
| A. niger 97-0626 (2 Hours) | 0.0598 | 2.019 | 33.8 |
| F. oxysporum | 0.054 | 0.973 | 18.0 |

From the data presented in this experiment it can be seen that the MTS/MEN combination is a useful indicator of A. fumigatis 8001, A. niger 97-0626 and F. oxysporum viability. For each of the organisms tested, there were significant differences between blank and spore containing wells (Table 10).

Other Embodiments

The present invention has been described in terms of particular embodiments found or proposed by the present inventors to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the invention.

What is claimed is:

1. A method for Quantitating the amount of living fungal cells in a culture, said method comprising the steps of:
a) contacting the culture with MTS and MEN so that any living fungal cells present in the culture will convert said MTS into a formazan reaction product, wherein the rate of said conversion is increased by the presence of said MEN; and
b) measuring said formazan reaction product spectrophotometrically as a measure of living fungus in said culture.

2. The method of claim 1, further comprising the step of c) contacting the culture, before, during, or following the addition of MTS and MEN, with a test substance to determine whether the test substance inhibits or promotes the growth or viability of said fungal cells.

3. The method of claim 2, further comprising (d) providing a control culture lacking said test substance, (e) spectrophotometrically measuring formazan production in said control culture, and (f) comparing formazan production in said test and control cultures.

4. The method of claim 2, wherein said method is conducted using multiple different test substances.

5. The method of claim 4, wherein said method is used in a high throughout mode.
